(19) United States
(12) Patent Application Publication
(10) Pub. No.: US 2025/0359837 A1
(43) Pub. Date: Nov. 27, 2025

(54) SIMULATING X-RAY FROM LOW DOSE CT

(71) Applicant: KONINKLIJKE PHILIPS N.V., EINDHOVEN (NL)

(72) Inventors: CHRISTIAN WUELKER, HAMBURG (DE); MICHAEL GRASS, BUCHHOLZ IN DER NORDHEIDE (DE); MERLIJN SEVENSTER, HAARLEM (NL); HILDO LAMB, LEIDEN (NL)

(21) Appl. No.: 18/867,585

(22) PCT Filed: May 22, 2023
(86) PCT No.: PCT/EP2023/063620

§ 371 Date: Nov. 20, 2024

Related U.S. Application Data
(60) Provisional application No. 63/344,852, filed on May 23, 2022.

(51) Int. Cl. A61B 6/00; A61B 6/03; G06T 3/4053; G06T 5/60; G06T 5/70; G06T 5/73; G06T 11/00; G06T 15/00

(52) U.S. Cl. CPC A61B 6/5223; A61B 6/5205; A61B 6/5258; G06T 3/4053; G06T 5/60; G06T 5/70; G06T 5/73; G06T 11/006; G06T 15/00; A61B 6/032; G06T 2200/04; G06T 2207/10081; G06T 2207/20084; G06T 2210/41

(57) ABSTRACT

Systems and methods for transforming three-dimensional computed tomography (CT) data into two dimensional images are provided. Such a method is provided including retrieving three-dimensional CT imaging data, where the three-dimensional CT imaging data comprises projection data acquired from a plurality of angles about a central axis. Once the three-dimensional CT imaging data is retrieved, the imaging data is processed as a three-dimensional image and the method proceeds to generate a two-dimensional image by tracing rays from a simulated radiation source outside of the three-dimensional image. The two-dimensional image is then presented to a user as a simulated X-Ray.

### FIELD

The present disclosure generally relates to systems and methods for simulating conventional X-ray images from CT data. In particular, the present disclosure relates to presenting ultra-low-dose CT data to users as a two-dimensional image simulating an X-ray image.

### BACKGROUND

Computed tomography (CT) imaging provides advantages over conventional planar X-ray (CXR) imaging. Accordingly, CT imaging has replaced X-ray imaging in various clinical settings and is increasingly adopted in additional clinical settings in place of such X-ray imaging.

This is particularly true in the case of low-dose and ultra-low-dose CT imaging (ULDCT), which now aims at replacing CXR in additional settings, such as routine chest imaging in outpatient settings.

One main advantage of CT imaging over CXR is that CT provides additional information and, in particular, three-dimensional spatial information. Also, CXR has a relatively low sensitivity and high false negative rate in many clinical scenarios. CT imaging, because of the additional information associated with it, is also more amenable to various image processing and AI based diagnosis techniques.

On the other hand, CXR has a higher spatial resolution and suffers less from noise than conventional CT imaging and, in particular, ULCT imaging.

One reason that CT imaging has not been more widely adopted in routine clinical settings is that reading time of CT imaging is substantially higher than CXR. This is partially because radiologists are more familiar with C×R images and are, therefore, more comfortable reading and basing diagnoses on such conventional planar X-ray images.

However, to the extent that radiologists continue to rely on C×R images, they are forgoing the advantages, both in terms of additional information and analytical capability, of CT imaging.

There is a need for a CT imaging system and method and, in particular, an ULDCT imaging system and method that can present data to radiologists in a form more easily interpreted and more likely to be adopted. There is a further need for such a system in which the advantages of CT imaging are made available to radiologists in an approachable and accessible way.

### SUMMARY

Systems and methods for transforming three-dimensional computed tomography (CT) data into two dimensional images are provided. Such a method is provided including retrieving three-dimensional CT imaging data, where the three-dimensional CT imaging data comprises projection data acquired from a plurality of angles about a central axis.

Once the three-dimensional CT imaging data is retrieved, the imaging data is processed as a three-dimensional image and the method proceeds to generate a two-dimensional image by tracing rays from a simulated radiation source outside of a subject of the three-dimensional image.

The two-dimensional image is then presented to a user as a simulated X-ray.

In some embodiments, the processing of the three-dimensional CT imaging data includes reconstructing the three-dimensional image using filtered back projection. In some such embodiments, the three-dimensional CT imaging data comprises ultra-low-dose CT imaging data, and processing the three-dimensional CT imaging data further comprises denoising the imaging data.

In some such embodiments, processing the three-dimensional CT imaging data further includes performing an Artificial Intelligence (AI) based super-resolution process. Such a super-resolution process may include a deblurring process.

In some embodiments, denoising the imaging data may comprise applying a trained convolutional neural network (CNN) to the three-dimensional CT imaging data.

In some embodiments, a denoising process is applied to the three-dimensional CT imaging data prior to reconstructing the three-dimensional image.

In some embodiments, the method further includes processing the two-dimensional image prior to presenting the image to the user by applying a style to the two-dimensional image. Such a style may be derived from a plurality of X-ray images, and the style may then modify the appearance of the two-dimensional image, but not the morphological contents of the two-dimensional image.

In some such embodiments, the plurality of X-ray images are conventional planar X-ray images.

In some embodiments, the processing of the three-dimensional CT imaging data includes identifying at least one physical element in the three-dimensional image and removing or masking out the at least one physical element from the three-dimensional image prior to generating the two-dimensional image.

In some such embodiments, the physical element is an anatomical element, such as ribs or a heart. In other embodiment, the physical element is a table or an implant.

In some embodiments, the two-dimensional image is presented to the user with the three-dimensional image, and an indicator is incorporated into the three-dimensional image indicating a segment of the three-dimensional image represented in the two-dimensional image.

In some embodiments, the method further includes processing the two-dimensional image prior to presenting the image to the user. Such processing may include applying a denoising or super-resolution process to the image.

In some embodiments, AI based denoising or super-resolution processes are applied in 2D planes in the three-dimensional CT imaging data.

In some embodiments, the three-dimensional CT imaging data comprises spectral data or photon-counting data, and the simulated X-ray is a simulated spectral X-ray or photon-counting X-ray.

In some embodiments, the generation of the two-dimensional image is performed by a neural network. In some such embodiments, neural network is a trained convolutional neural network.

### BRIEF DESCRIPTION OF THE DRAWINGS

FIG. 1 is a schematic diagram of a system according to one embodiment of the present disclosure.

FIG. 2 illustrates an exemplary imaging device according to one embodiment of the present disclosure.

FIG. 3 illustrates a schematic workflow for implementing a method according to one embodiment of the present disclosure.

FIG. 4 is a flow chart illustrating a method according to one embodiment of the present disclosure.

FIG. 5 schematically shows a ray tracing process applied to a three-dimensional image usable in the context of one embodiment of the present disclosure.

FIGS. 6A-C illustrate an implementation of a style transfer for use in the method according to one embodiment of the present disclosure.

### DETAILED DESCRIPTION OF THE PREFERRED EMBODIMENTS

The description of illustrative embodiments according to principles of the present disclosure is intended to be read in connection with the accompanying drawings, which are to be considered part of the entire written description. In the description of embodiments of the disclosure disclosed herein, any reference to direction or orientation is merely intended for convenience of description and is not intended in any way to limit the scope of the present disclosure. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the disclosure are illustrated by reference to the exemplified embodiments. Accordingly, the disclosure expressly should not be limited to such exemplary embodiments illustrating some possible non-limiting combination of features that may exist alone or in other combinations of features; the scope of the disclosure being defined by the claims appended hereto.

This disclosure describes the best mode or modes of practicing the disclosure as presently contemplated. This description is not intended to be understood in a limiting sense, but provides an example of the disclosure presented solely for illustrative purposes by reference to the accompanying drawings to advise one of ordinary skill in the art of the advantages and construction of the disclosure. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality.

Both computed tomography (CT) and conventional planar X-ray (CXR) are used in medical imaging. However, CT imaging and, in particular, ultra-low-dose CT imaging (ULDCT) aim at replacing CXR in many clinical settings such as, for example, chest imaging in routine outpatient settings.

Some of the main advantages of ULDCT imaging are immediately apparent. CT imaging, including ULDCT, provides three-dimensional spatial information, which allows for sophisticated analytical techniques. Further, ULDCT avoids the relatively low sensitivity and high false negative rates associated with CXR in many clinical scenarios.

However, ULDCT has a slower read time than CXR, and radiologists are less familiar and less comfortable with ULDCT. As such, radiologists prefer to be presented with and make diagnoses based on more familiar C×R images. The methods described herein therefore provide a workflow for generating artificial C×R images, or images stylized to have the appearance of C×R images, from ULDCT data. Such methods may be implemented or enhanced using artificial intelligence (AI) techniques, including the use of learning algorithms in the form of neural networks, such as convolutional neural networks (CNN).

Accordingly, methods are provided for transforming three-dimensional CT data into two-dimensional images. In this way, CXR style images may be generated from ULDCT data and presented to radiologists. Such presentation may follow the implementation of analytical techniques to the underlying ULDCT data in either raw or three-dimensional image formats, and may be presented to radiologists either as a proxy for a C×R image or in the context of a corresponding ULDCT based image interface.

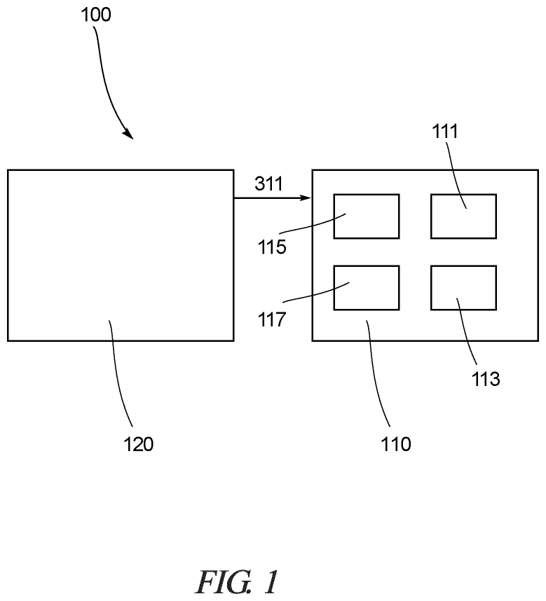

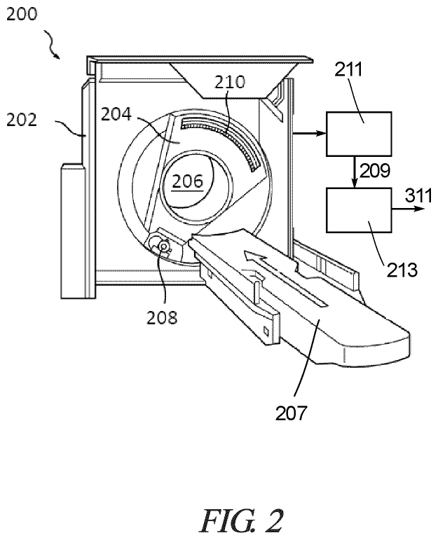

Accordingly, ULDCT imaging data may be generated as three-dimensional CT imaging data using a system such as that illustrated in FIG. 1 and by way of an imaging device such as that illustrated in FIG. 2. The retrieved data may then be processed using the processing device of the system of FIG. 1.

FIG. 1 is a schematic diagram of a system 100 according to one embodiment of the present disclosure. As shown, the system 100 typically includes a processing device 110 and an imaging device 120.

The processing device 110 may apply processing routines to images or measured data, such as projection data, received from the image device 120. The processing device 110 may include a memory 113 and processor circuitry 111. The memory 113 may store a plurality of instructions. The processor circuitry 111 may couple to the memory 113 and may be configured to execute the instructions. The instructions stored in the memory 113 may comprise processing routines, as well as data associated with processing routines, such as machine learning algorithms, and various filters for processing images.

The processing device 110 may further include an input 115 and an output 117. The input 115 may receive information, such as three-dimensional images or measured data, such as three-dimensional CT imaging data, from the imaging device 120. The output 117 may output information, such as filtered images, or converted two-dimensional images, to a user or a user interface device. The output may include a monitor or display.

In some embodiments, the processing device 110 may relate to the imaging device 120 directly. In alternate embodiments, the processing device 110 may be distinct from the imaging device 120, such that the processing device 110 receives images or measured data for processing by way of a network or other interface at the input 115.

In some embodiments, the imaging device 120 may include an image data processing device, and a spectral or conventional CT scanning unit for generating CT projection data when scanning an object (e.g., a patient). In some embodiments, the imaging device 120 may be a conventional CT scanning unit configured for generating helical scans.

FIG. 2 illustrates an exemplary imaging device 200 according to one embodiment of the present disclosure. It will be understood that while a CT imaging device 200 is shown, and the following discussion is generally in the context of CT images, similar methods may be applied in the context of other imaging devices, and images to which these methods may be applied may be acquired in a wide variety of ways.

In an imaging device 200 in accordance with embodiments of the present disclosure, the CT scanning unit may be adapted for performing one or multiple axial scans and/or a helical scan of an object in order to generate the CT projection data. In an imaging device 200 in accordance with embodiments of the present disclosure, the CT scanning unit may comprise an energy-resolving photon counting or spectral dual-layer image detector. Spectral content may be acquired using other detector setups as well. The CT scanning unit may include a radiation source that emits radiation for traversing the object when acquiring the projection data.

In the example shown in FIG. 2, the CT scanning unit 200, e.g. the Computed Tomography (CT) scanner, may include a stationary gantry 202 and a rotating gantry 204, which may be rotatably supported by the stationary gantry 202. The rotating gantry 204 may rotate about a longitudinal axis around an examination region 206 for the object when acquiring the projection data. The CT scanning unit 200 may include a support 207 to support the patient in the examination region 206 and configured to pass the patient through the examination region during the imaging process.

The CT scanning unit 200 may include a radiation source 208, such as an X-ray tube, which may be supported by and configured to rotate with the rotating gantry 204. The radiation source 208 may include an anode and a cathode. A source voltage applied across the anode and the cathode may accelerate electrons from the cathode to the anode. The electron flow may provide a current flow from the cathode to the anode, such as to produce radiation for traversing the examination region 206.

The CT scanning unit 200 may comprise a detector 210. The detector 210 may subtend an angular arc opposite the examination region 206 relative to the radiation source 208. The detector 210 may include a one- or two-dimensional array of pixels, such as direct conversion detector pixels. The detector 210 may be adapted for detecting radiation traversing the examination region 206 and for generating a signal indicative of an energy thereof.

The CT scanning unit 200 may include generators 211 and 213. The generator 211 may generate tomographic projection data 209 based on the signal from the detector 210. The generator 213 may receive the tomographic projection data 209 and, in some embodiments, generate three-dimensional CT imaging data 311 of the object based on the tomographic projection data 209. In some embodiments, the tomographic projection data 209 may be provided to the input 115 of the processing device 110, while in other embodiments the three-dimensional CT imaging data 311 is provided to the input of the processing device.

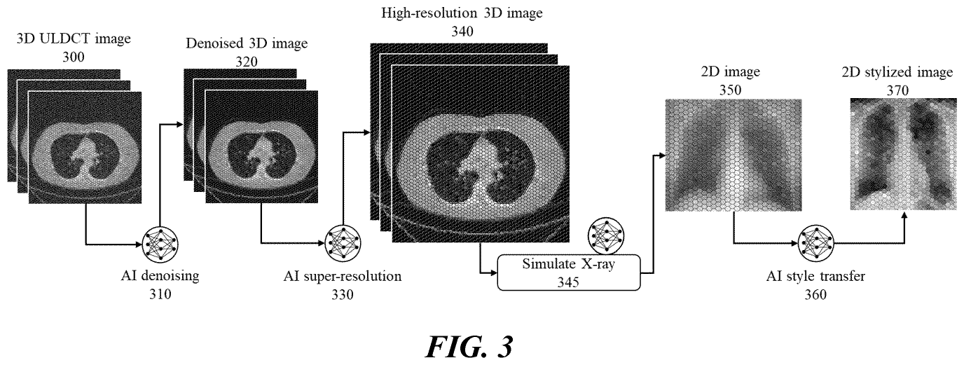

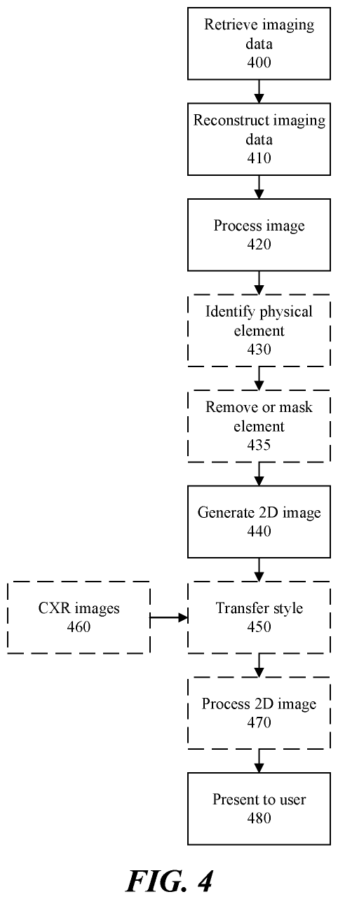

FIG. 3 illustrates a schematic workflow for implementing a method in accordance with one embodiment of the present disclosure. FIG. 4 is a flow chart illustrating a method in accordance with one embodiment of the present disclosure. As shown, the method typically includes first retrieving (400) three-dimensional CT imaging data. Such three-dimensional CT imaging data comprises projection data for a subject acquired from a plurality of angles about a central axis.

Accordingly, in the context of the imaging device 200 of FIG. 2, the subject may be a patient on the support 207, and the central axis may be an axis passing through the examination region. Where the three-dimensional CT imaging data is acquired from the imaging device 200, the rotating gantry 204 may then rotate about the central axis of the subject, thereby acquiring the projection data from various angles.

Once acquired, the three-dimensional CT imaging data 311 is reconstructed (410) as a three-dimensional image 300 in preparation for processing. The three-dimensional CT imaging data 311 is then processed (420) as a three-dimensional image 300.

It is understood that while the reconstruction (at 410) and the processing (at 420) are indicated as distinct processes, the reconstruction itself may be the actual processing of the three-dimensional CT imaging data as a three-dimensional image. Similarly, the reconstruction may be part of such processing. Such reconstruction (at 410) may be by using standard reconstruction techniques, such as by way of filtered back projection.

As shown in FIG. 3, the processing may include denoising 310 which may be, for example, by way of a neural network or other artificial intelligence based learning algorithm. In the example shown, the denoising 310 is by way of a convolutional neural network (CNN) previously trained on appropriate images. Such denoising processes 310 may be utilized, for example, where the CT imaging is noisy, as in the case of ULDCT images. The denoising process may then result in a denoised or partially denoised three-dimensional image 320.

The denoising process 310 described may be a process that incorporates features that allow it to generalize well to different contrasts, anatomies, reconstruction filters, and noise levels. Such a denoising process 310 may compensate for the high noise levels inherent in ULDCT images.

In the example shown, the processing of the three-dimensional CT images (at 420) may further include the implementation of a super-resolution process 330. As in the case of the denoising process 310, the super-resolution process 330 may be by way of AI based learning algorithm, such as a CNN. In some embodiments, the super-resolution process 330 may include deblurring of the image. The super-resolution process 330 may then result in a higher resolution three-dimensional image 340.

The super-resolution process 330 typically interpolates the image to smaller voxel sizes while maintaining perceived image sharpness or improving perceived image sharpness. AI based super-resolution processes 330 may be trained on either real CT images, including ULDCT images, or more generic image material, such as natural high-resolution photos.

In the embodiment shown, both denoising processes 310 and super-resolution processes 330 are applied in sequence. However, it will be understood that both processes may be incorporated into a single neural network, such as a CNN. Further, while both processes 310, 330 are shown as applied to the three-dimensional image 300, in some embodiments, the processes may be applied directly to the three-dimensional CT imaging data prior to reconstruction (at 410). Further, in some embodiments, one or both processes 310, 330 may be applied on two-dimensional planes in the three-dimensional CT imaging data set perpendicular to the projection direction to be used to generate the two-dimensional image discussed below.

In some embodiments, processing may further comprise identifying (430) at least one physical element in the three-dimensional image. Once identified (at 430), the physical element may be removed or masked out (435) of the three-dimensional image. By removing or masking out (435) an element prior to the generation of a two-dimensional image, such a physical element may be removed from a simulated X-ray to be generated from the CT imaging data.

The physical element identified (at 430) may be an anatomical element, such as one or more ribs or a heart. By removing such anatomical element from a simulated X-ray, other anatomical elements of interest to a radiologist viewing the images may be more easily visible, and the simulated X-ray may show a cutaway of a patient's chest cavity without interfering ribs, for example.

Alternatively, the physical element identified (at 430) may be a table 207 or an implant. CT imaging data is typically acquired from a patient lying on a table or other support 207, as in the imaging device 200 discussed above. In contrast, conventional planar X-rays are often acquired from standing patients. Accordingly, by removing a support 207, a simulated X-ray may appear more natural to a radiologist viewing the images. Similarly, removing an implant may provide a better view of a patient's anatomy.

In some embodiments, rather than removing the physical element identified (at 430), the physical element may instead be weighted. Similarly, different sections of the three-dimensional image 300 may be weighted differently.

Following the processing of the three-dimensional CT imaging data (at 420), the method proceeds to generate a two-dimensional image 350 by tracing rays from a simulated radiation source outside of a subject of the three-dimensional image (440). Such a ray tracing process may be, for example, by implementing a Siddons-Jacobs ray-tracing algorithm.

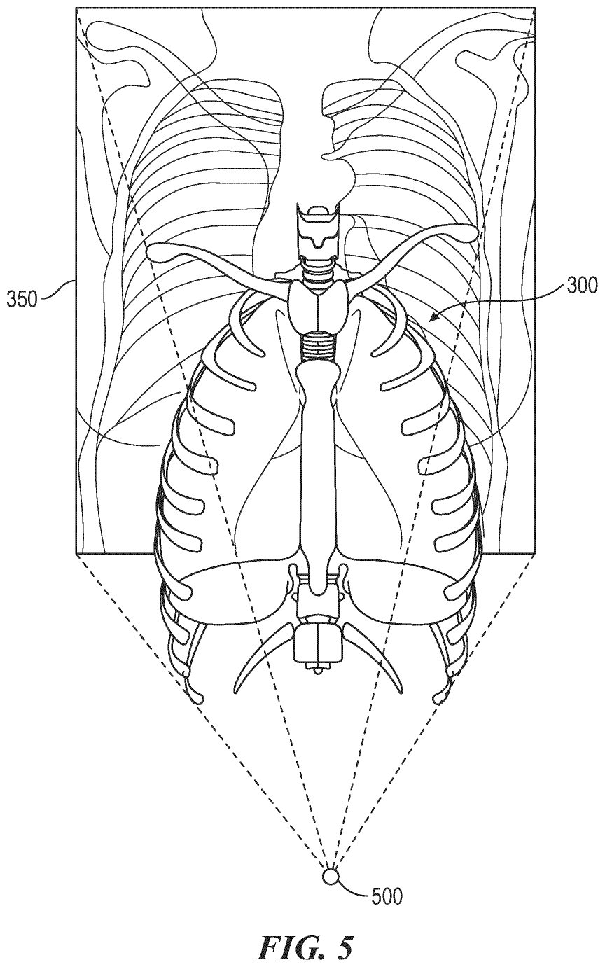

FIG. 5 illustrates an implementation of a ray tracing process (440) to a three-dimensional image 300 in order to generate a two-dimensional image 350. The ray tracing process may then proceed by simulating the process of an X-ray 345 by propagating incident X-ray photons from a simulated radiation source 500 through the reconstructed three-dimensional image 300. The generation of the two-dimensional image 350 may be by way of a neural network, such as a CNN, and in such cases, the CNN may incorporate one or more of denoising, super-resolution, or style transfer processes discussed elsewhere herein. Such a neural network may be a generative adversarial network (GAN). In such an embodiment, many or all of the steps described herein may be incorporated into a single network, such that CT volume data is provided to the network, and simulated CXR projections are output.

In some embodiments, the projection angle or orientation of the ray tracing process (440) may be adjusted in order to improve the resulting two-dimensional image 350. Similarly, weighting of physical elements in the three-dimensional image 300 may be adjusted in order to improve the resulting two-dimensional image 350.

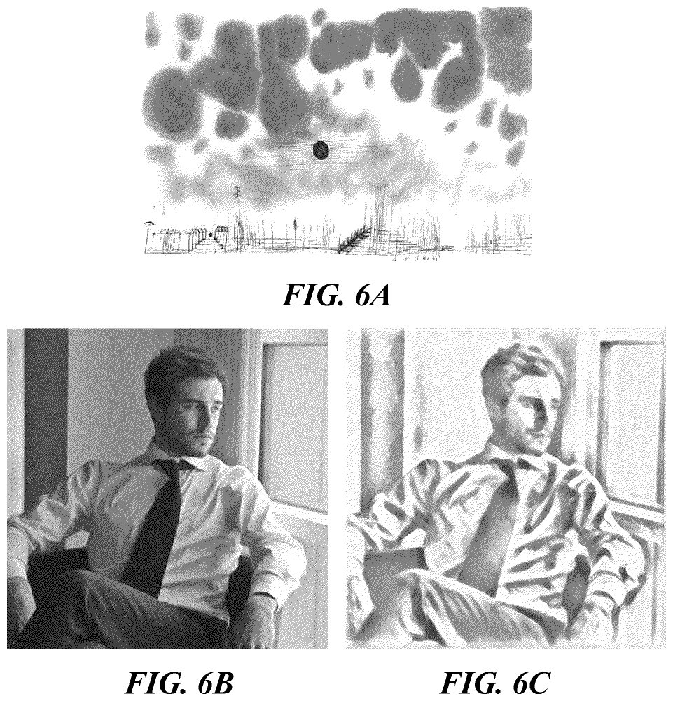

Once the two-dimensional image 350 has been generated, in some embodiments, an optional style transfer process (450) may be applied, where a style is applied to the two-dimensional image. In such embodiments, the style applied (at 450) may be derived from a plurality of X-ray images (460), such as conventional planar X-ray images, and may be applied by way of an AI algorithm 360, such as a CNN. Such a style modifies the appearance of the two-dimensional image, but not the morphological contents of the underlying image. Such a process is discussed in more detail below in reference to FIG. 6 and may be used to generate a second two-dimensional image 370 in the “style” of a conventional X-ray.

In some embodiments, further processing may be applied to the two-dimensional image (470) following the ray tracing process (at 440). Such processing may include a denoising or super-resolution process applied to the image, and may be in place of or in addition to the application of such processes to the three-dimensional image.

Following any processing (at 470), the two-dimensional image 350 or stylized image 370 may be presented to a user (480), such as a radiologist. Such a presentation (at 480) may comprise presenting the image as if it were a conventional planar X-ray, or it may comprise incorporating the two-dimensional image 350 or stylized image 370 into a user interface with the three-dimensional image 300. For example, in some embodiments, the two-dimensional image 350 or stylized image 370 may be presented to the user with the three-dimensional image 300 and with an indicator incorporated into the three-dimensional image indicating a segment of the three-dimensional image represented in the two-dimensional image. Accordingly, the two-dimensional image 350 or stylized image 370 may be presented as a section view of the three-dimensional image 300, with the three-dimensional image contextualizing the section. The two-dimensional image 350 or stylized image 370 may then be used as an avatar to guide ULDCT reading, and AI feedback may be projected onto the two-dimensional image in order to help radiologists quickly identify problem areas to review and report in detail on the original ULDCT images.

In some embodiments, the three-dimensional CT imaging data may comprise spectral data. In such embodiments, the simulated X-ray may similarly simulate a spectral X-ray. Similarly, the method may be applied to photon counting or phase contract CT data sets, and the same may then be reflected in the resulting two-dimensional images.

Similarly, while the method is described in the context of CT data, and in particular, ULDCT data, similar methods may be applied to magnetic resonance (MR) image data by applying MR-CT image translation and subsequently applying the method steps described.

FIGS. 6A-C illustrate an implementation of a style transfer for use in the method of the present disclosure. As shown, a set of “style images,” such as that shown in FIG. 6A, may be used to define a certain style of an image. An AI algorithm, such as a CNN, may then be trained to apply a style derived from the “style images” to a received image.

Accordingly, when an image, such as that shown in 6B, is received by the AI algorithm, the image may then be re-rendered and output in the style of the “style images,” as shown in FIG. 6C. In the examples shown, a style may be derived from a specific artist, in this case Paul Klee, and applied to a generic portrait.

In the embodiments discussed herein, the “style images” used to train the AI algorithm may be, for example, conventional X-ray (CXR) images. In this way, the two-dimensional images 350 generated from the ULDCT three-dimensional images 300 may be transformed to appear more like C×R images. Such stylized images 370 may then be used in practice. It is noted that a style transfer does not change the morphological contents of an image, instead changing only its appearance. As such, the style transfer described is a conservative technique.

Many of the steps of the methods described herein may be implemented as AI methods, such as CNNs. Such methods may be used with variable strength of effect, allowing for different denoising levels or super-resolution levels, for example.

The methods according to the present disclosure may be implemented on a computer as a computer implemented method, or in dedicated hardware, or in a combination of both. Executable code for a method according to the present disclosure may be stored on a computer program product. Examples of computer program products include memory devices, optical storage devices, integrated circuits, servers, online software, etc. Preferably, the computer program product may include non-transitory program code stored on a computer readable medium for performing a method according to the present disclosure when said program product is executed on a computer. In an embodiment, the computer program may include computer program code adapted to perform all the steps of a method according to the present disclosure when the computer program is run on a computer. The computer program may be embodied on a computer readable medium.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A method for transforming three-dimensional computed tomography (CT) data into two-dimensional images, comprising:
retrieving three-dimensional CT imaging data, the three-dimensional CT imaging data comprising projection data acquired from a plurality of angles about a central axis;
processing the three-dimensional CT imaging data as a three-dimensional image;
generating a two-dimensional image by tracing rays from a simulated radiation source outside of a subject of the three-dimensional image; and
presenting the two-dimensional image to a user as a simulated X-ray.

2. The method of claim 1, wherein processing the three-dimensional CT imaging data comprises reconstructing the three-dimensional image using filtered back projection.

3. The method of claim 2, wherein the three-dimensional CT imaging data comprises ultra-low-dose CT imaging data, and wherein processing the three-dimensional CT imaging data comprises denoising the imaging data.

4. The method of claim 3, wherein processing the three-dimensional CT imaging data further comprises performing an AI based super-resolution process.

5. The method of claim 4 wherein the super-resolution process comprises a deblurring process.

6. The method of claim 3, wherein denoising the imaging data comprises applying a trained convolutional neural network (CNN) to the three-dimensional CT imaging data.

7. The method of claim 2, wherein a denoising process is applied to the three-dimensional CT imaging data prior to reconstructing the three-dimensional image.

8. The method of claim 1 further comprising processing the two-dimensional image prior to presenting the two-dimensional image to the user by applying a style to the two-dimensional image, the style derived from a plurality of X-ray images, and wherein the style modifies the appearance of the two-dimensional image but not the morphological contents of the two-dimensional image.

9. The method of claim 8 wherein the plurality of X-ray images are conventional planar X-ray images.

10. The method of claim 1 wherein processing the three-dimensional CT imaging data comprises identifying at least one physical element in the three-dimensional image and removing or masking out the at least one physical element from the three-dimensional image prior to generating the two-dimensional image.

11. The method of claim 10 wherein the at least one physical element is an anatomical element.

12. The method of claim 1 wherein the two-dimensional image is presented to the user with the three-dimensional image, and wherein an indicator is incorporated into the three-dimensional image indicating a segment of the three-dimensional image represented in the two-dimensional image.

13. The method of claim 1 further comprising processing the two-dimensional image prior to presenting the two-dimensional image to the user, wherein the processing of the two-dimensional image comprises applying a denoising or super-resolution process to the image.

14. The method of claim 1 further comprising performing AI based denoising or super-resolution processes in 2D planes in the three-dimensional CT imaging data.

15. The method of claim 1, wherein the three-dimensional CT imaging data comprises spectral data or photon-counting data, and wherein the simulated X-ray is a simulated spectral X-ray or photon counting X-ray.

16. The method of claim 1, wherein the generation of the two-dimensional image is performed by a neural network.

17. A system for transforming three-dimensional computed tomography (CT) data into two-dimensional images, comprising:
a memory that stores a plurality of instructions; and
processor circuitry that couples to the memory and is configured to execute the plurality of instructions to:
retrieve three-dimensional CT imaging data, the three-dimensional CT imaging data comprising projection data acquired from a plurality of angles about a central axis;
process the three-dimensional CT imaging data as a three-dimensional image;
generate a two-dimensional image by tracing rays from a simulated radiation source outside of a subject of the three-dimensional image; and
present the two-dimensional image to a user as a simulated X-ray.

18. A non-transitory computer-readable medium for storing executable instructions, which cause a method to be performed to transform three-dimensional computed tomography (CT) data into two-dimensional images, the method comprising:
retrieving three-dimensional CT imaging data, the three-dimensional CT imaging data comprising projection data acquired from a plurality of angles about a central axis;
processing the three-dimensional CT imaging data as a three-dimensional image;
generating a two-dimensional image by tracing rays from a simulated radiation source outside of a subject of the three-dimensional image; and
presenting the two-dimensional image to a user as a simulated X-ray.
